(19) United States
(12) Patent Application Publication
(10) Pub. No.: US 2013/0165398 A1
(43) Pub. Date: Jun. 27, 2013

(54) CONCENTRATION-ENHANCING DRINK

(71) Applicant: Johannes Huber, Vienna (AT)

(72) Inventor: Johannes Huber, Vienna (AT)

(73) Assignee: PAUL HOCHEGGER, WIEN (AT)

(21) Appl. No.: 13/638,741

(22) PCT Filed: Mar. 29, 2011
(86) PCT No.: PCT/EP11/54825

§ 371 Date: Mar. 4, 2013

(30) Foreign Application Priority Data
Mar. 29, 2010 (EP) 101 58 180.9

(51) Int. Cl. A61K 31/353; A61K 31/405; A61K 31/706

(52) U.S. Cl. CPC A61K 31/353; A61K 31/706; A61K 31/405
USPC 514/43; 514/419; 514/783

(57) ABSTRACT

A concentration-enhancing drink contains, in each case independently of one another, per liter: 1 mg to 10 g, preferably 1 to 1000 mg, still more preferably 10 to 500 mg, in particular 50 to 300 mg, of epigallocatechin gallate; 1 to 500 mg, preferably 10 to 100 mg, in particular 20 to 80 mg, of nicotinamide ribose; and/or 0.05 to 10 g, preferably 0.1 to 5 g, in particular 0.5 to 3 g of tryptophan.

The present invention relates to concentration-enhancing compositions.

“Concentration” denotes the deliberate focusing of attention on a certain activity, achievement of a target achievable in the short term, or solving of a stated problem. In this case, “deliberate focusing” is taken to mean that a person consciously pays attention for a certain time to that which has just been carried out (or is to be carried out) or perceived. A concentrated activity is associated with mental stress, but where the degree of concentration generally decreases with time, and the “level of attention” falls.

A “concentration deficit” is generally taken to mean an impairment of the ability to keep attention directed towards a certain (mental) activity. Concentration deficit is used, chiefly, in medicine, in psychotherapy and in pedagogics as a description of symptoms.

Concentration and concentration deficits are generally measured or observed scientifically using standardized tests (attention-stress tests).

Factors affecting concentration include the physical and psychic state of the person in question, in particular depending on nutrition, environmental conditions and social components. Concentration deficits in this case can be due to neurological, psychosomatic or organic causes.

In the prior art, very many ways and means are known in order to increase concentration, to maintain the level of attention as long as possible, or to remedy or eliminate concentration deficits.

For instance, it is known that napping, relaxing, moderate endurance sport or concentration exercises act beneficially on concentration and maintaining the level of attention, whereas stress or lack of sleep cause concentration deficit. It is likewise described that a balanced glucose level can act beneficially on concentration, whereas a very low glucose level can make concentration more difficult.

Numerous liquid and solid foods are also described to which concentration-enhancing effects are ascribed, e.g. omega-3 fatty acids (fish oil), vitamins, chiefly vitamin B, Lentaya, caffeine, ginseng, guarana, ginkgo, lecithin, curcumin, etc.

It is an object of the present invention to provide a concentration-enhancing drink.

Accordingly the present invention relates to a drink containing, in each case independently of one another, per l
1 mg to 10 g, preferably 1 to 1000 mg, still more preferably 10 to 500 mg, in particular 50 to 300 mg, epigallocatechin gallate (EGCG),
1 to 500 mg, preferably 10 to 100 mg, in particular 20 to 80 mg, nicotinamide-ribose (NR) and/or 0.05 to 10 g, preferably 0.1 to 5 g, in particular 0.5 to 3 g, tryptophan.

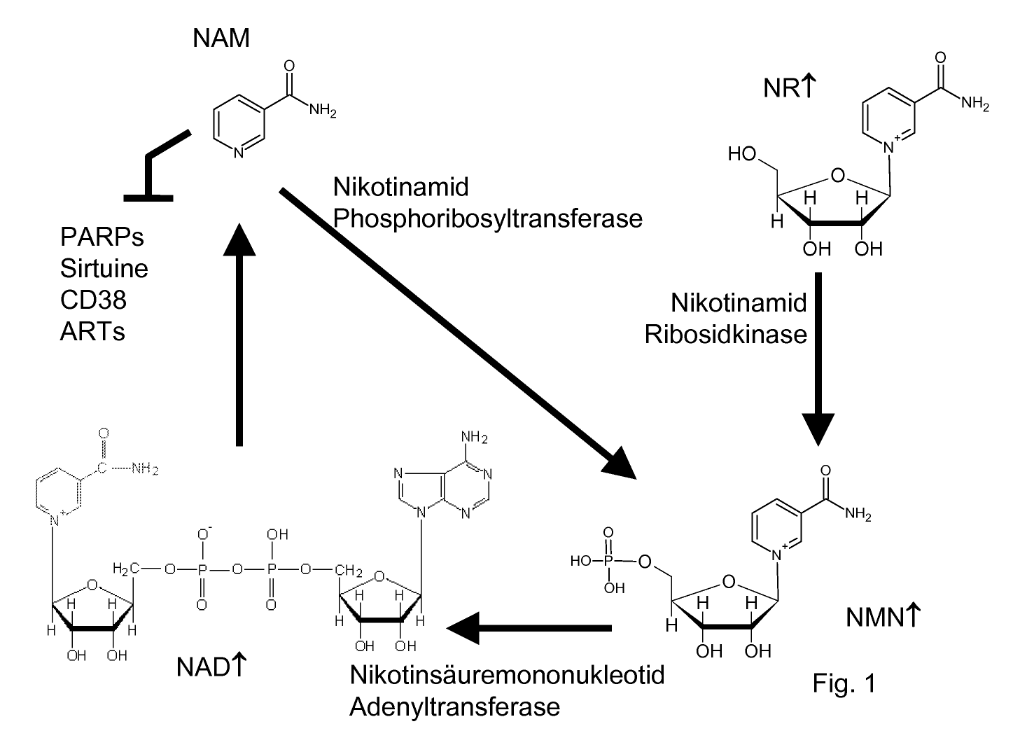

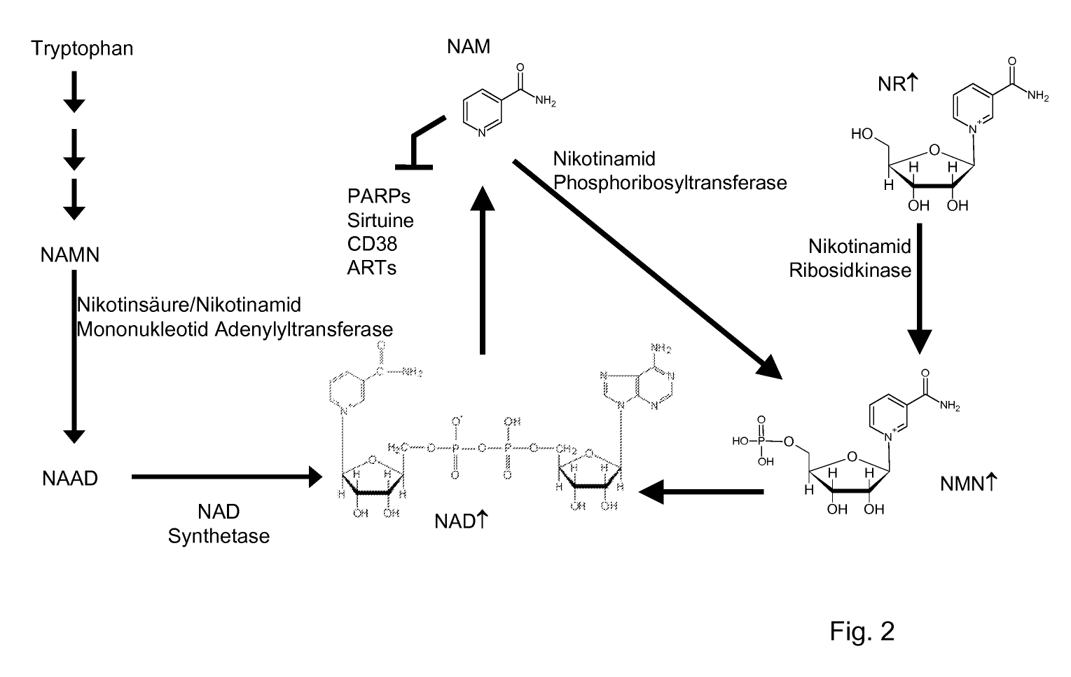

Epigallocatechin gallate (EGCG; (2R,3R)-2-(3,4,5-trihydroxyphenyl)-3,4-dihydro-1(2H)-benzopyran-3,5,7-triol 3-(3,4,5-trihydroxybenzoate)) is a naturally occurring antioxidant. EGCG is a catechin that belongs to the subgroup of polyphenols and occurs in green tea. In addition to many other activities, EGCG exhibits an insulin-depressing effect and also an activity in enhancing concentration. EGCG increases the endogenous formation of nicotinamide adenine dinucleotide (NAD)=NAD⁺)). By administering EGCG, the nicotinamide riboside kinase is excited and beta-nicotinamide mononucleotide is formed to an increased extent. Tryptophan consumed with the diet is converted in the cell into nicotinic acid mononucleotide (NAMN), which is again converted by nicotinic acid/nicotinamide mononucleotide adenyl transferase into nicotinic acid adenine dinucleotide (NAAD), from which NAD is formed via NAD synthase. EGCG stimulates NAD synthase and also nicotinic acid/nicotinamide mononucleotide adenyl transferase, whereby with the simultaneous presence of tryptophan endogenous formation of NAD is increased (see FIGS. 1 and 2). EGCG is preferably provided as an extract from biological sources in the drink according to the invention, particularly preferably as an extract from tea plants, in particular obtained directly from green tea or by the Matcha process.

Compositions which contain EGCG either as a pure substance or as a component of green tea and which are used, inter alia, for treatment of mental disorders or nervous disorders are described, inter alia, in JP 203/286167 A, JP 11/018,722 A, US 2002/086067 A1, US 2008/213401 A1 and WO 2008/006082 A2.

Nicotinamide-ribose (NR; nicotinamide riboside) (1-[3,4-dihydroxy-5-(hydroxymethyl)oxolan-2-yl]pyridine-5-carboxamide; also termed “nicotinamide-riboside”, “nicotinamide-beta-riboside”, “nicotinamide ribonucleoside” “1-[2R,3R,4S,5R)-3,4-dihydroxy-5-(hydroxymethyl)oxolan-2-yl]pyridine-5-carboxamide”, “N-ribosylnicotinamide” (CAS: 1341-23-7)) is also a precursor for NAD and is converted by nicotinamide-ribose kinase (nrk) into nicotinamide mononucleotide, which itself is converted via nicotinamide phosphoribosyl transferase (NNAD) into NAD. Both nicotinamide-ribose kinase and nicotinic acid-nicotinamide-mononucleotide-adenyl transferase are stimulated by EGCG, in such a manner that the simultaneous administration of either tryptophan or nicotinamide-ribose with EGCG leads to an amplified endogenous NAD synthase. At the same time, the activity of the sirtuins, which is NAD-dependent, is stimulated thereby. Therefore, the activity of nicotinamide-riboside kinase is associated with the activity of sirtuins (especially Sirt 2). In the course of the present invention, it was also possible to obtain findings on NR in cellular NAD production, which findings show NR to be one of the central compounds in this synthesis.

Tryptophan ((S)-2-amino-3-(1H-indol-3-yl)propanoic acid) is an amino acid essential for humans.

As with EGCG, NR and tryptophan can also be used according to the invention both in the form of natural extracts and in synthetic form (cf. e.g. Yang et al., J. Med. Chem. 50 (2007): 6458-6461); another advantageous variant is the enzymatic production of the components, in particular of NR. Preference is given in all cases (e.g. from natural sources, from formulations of chemical syntheses or products of enzymatic syntheses (or combinations of same)) to purified formulations as starting materials for producing the drink according to the invention (e.g. such extracts or formulations which contain EGCG and NR and/or tryptophan at at least 10% by weight, preferably at at least 30% by weight, in particular at at least 50% by weight, in each case based on dry weight). For example, NR occurs in some foods, especially in milk, but can also be supplied as a dietetic food. All quantitative statements made herein or percentages relate to the respective compounds in pure form in the respective composition (unless explicitly stated otherwise).

The present invention is based on the concentration-enhancing activity of a combination of an effective amount of EGCG on the one hand, and an effective amount of NR or tryptophan or a combination of NR and tryptophan on the other, for providing a composition for concentration enhancement. The concentration enhancement achieved according to the invention can result, e.g., as an enhancement of the relative concentration capacity of an individual person (compared with the concentration capacity of the same person before intake of the composition according to the invention) or as an improvement in mental stamina (e.g. delay of the drop in concentration in the case of continued concentrated activities). Accordingly, the present invention is suitable, especially, for concentration enhancement in the case of mental activities, in particular in the case of relatively long-lasting activities which demand a high concentration capacity over a relatively long time. The composition according to the invention is particularly readily usable in teaching and educational activities (therefore for schoolchildren, students, teachers), but also for specialized occupations, such as air traffic control and traffic monitoring, quality control, work at computer screens etc. The present invention is also readily usable for free time activities which demand a certain level of concentration, e.g. for concentration-intense games (chess, computer games etc.). Using the present invention, not only can the individual concentration capacity be enhanced, a drop in performance in long-lasting activities can also be delayed or prevented, or this drop in performance can at all events be significantly reduced in extent. The invention is of course primarily conceived for application with humans, but in principle it is also possible for it to be applied to animals, e.g. higher mammals, since here the same conditions in the synergistic metabolic function of the combination according to the invention are present.

With the combination of EGCG and NR and/or tryptophan, according to the invention a composition is provided which has concentration-enhancing activity. Using the combination according to the invention, also, the concentration level can be prolonged. In addition, using the drink according to the invention, concentration deficits can also be remedied or even overcome.

Preferably, a sweetener is also added to the drink according to the invention. A preferred variant contains in this case the sweetener stevioside (19-O-β-D-glucopyranosyl-13-O-[β-D-glucopyranosyl(1→2)-β-D-glucopyranosyl]-steviol). Stevioside is a diterpene glycoside and is three hundred times sweeter than sugar. Stevioside can be obtained from stevia leaves (Stevia rebaudiana) and is suitable for diabetics. In the drink according to the invention, as sweetener, preferably Stevia (rebaudiana) or a stevia extract is added which can be treated enzymatically (by fermentation).

In the drink according to the invention, preferably 1 to 300 mg/l of stevioside are provided. Stevia leaves contain eight main glycosides (stevioside, steviolbioside, rebaudioside A, C, D, E and F and also dulcoside A), wherein stevioside at six to eighteen percent has the greatest proportion of the active ingredients found in stevia leaves. Four of these steviol glycosides (stevioside, rebaudioside A, rebaudioside C and dulcoside) are substantially responsible for the sweetening activity; among these, rebaudioside A has the best sensory properties of all four main glycosides since it is the sweetest and is not very bitter. If enzymatically obtained stevia products are provided according to the invention as sweeteners, they contain virtually 100% rebaudiosides and stevioside only in small traces. It is advantageous in this case that the drink then does not have a bitter off-taste or after-taste. Rebaudioside A is provided as sweetener in the drink according to the invention preferably from 0.5 mg to 200 mg (e.g. when enzymatically obtained stevia products are used for sweetening). However, it is preferred that the composition according to the invention is administered sugar-free, since adverse interactions with sugar (glucose, sucrose) are thereby prevented.

Preferably, the drink according to the invention contains further auxiliaries and flavor substances. In this case, especially, the flavor substances from natural sources known to those skilled in the art are used, e.g. fruit juices, fruit extracts, teas etc.

In association with EGCG and NR and/or tryptophan, stevioside prevents the hyperinsulinemic activity and thereby supports the effect of EGCG from another side.

The drink according to the invention can be provided in all customary drinks containers and in volumes customary on the market. According to preferred practical embodiments, the drinks according to the invention have a volume from 100 ml to 2 l, preferably from 250 ml to 1 l, in particular from 330 ml to 600 ml. Preferred retail volumes are 100, 200, 250, 330 and 500 ml; the 750 ml, 1 l and 2 l containers are suitable as large packs.

According to a variant of the present invention, the drink according to the invention can also be provided in dry form, in such a manner that it can be reconstituted using liquids (generally water (in particular mineral water), but fruit juices, sodas and the like are also suitable) to give the ready-to-use form. Preferably, the dry form is then packaged in single doses, for example as daily doses. Accordingly, daily doses according to the invention contain (e.g. in sachets, effervescent tablets etc.), for example (also in each case independently of one another), 1 mg to 10 g, preferably 1 to 1000 mg, still more preferably 10 to 500 mg, in particular 50 to 300 mg, EGCG, 1 to 500 mg, preferably 10 to 100 mg, in particular 20 to 80 mg, NR and/or 0.05 to 10 g, preferably 0.1 to 5 g, in particular 0.5 to 3 g, tryptophan and also optionally 0.1 mg to 500 mg, preferably 0.5 mg to 200 mg, in particular 1 to 100 mg, stevioside. Such a dry form (that is to say a formulation in solid form, in particular in powder form) can also be provided e.g. in the form of effervescent tablets. Appropriate auxiliaries and formulation substances or carrier substances that are typically usable are known to those skilled in the art in this field and can be correspondingly used if required in the compositions according to the invention. The drinks according to the invention can also be provided on the basis of highly concentrated concentrates (e.g. syrups) which can then be diluted in accordance with instructions, e.g. 1:2 to 1:20, preferably 1:5 to 1:10, in order to arrive at the values according to the invention of the concentrations in the drink.

The abovementioned drinks can preferably also already contain these daily doses as a retail unit, in such a manner that in one retail unit (100 ml, 200 ml, 250 ml, 330 ml, 500 ml, 750 ml, 1000 ml or other usual sizes for bottles, cans, drinks packs etc.) the daily dose (for example (in each case independently of one another) 1 mg to 10 g, preferably 1 to 1000 mg, still more preferably 10 to 500 mg, in particular 50 to 300 mg, EGCG, 1 to 500 mg, preferably 10 to 100 mg, in particular 20 to 80 mg, NR and/or 0.05 to 10 g, preferably 0.1 to 5 g, in particular 0.5 to 3 g, tryptophan and also optionally 0.1 mg to 500 mg, preferably 0.5 mg to 200 mg, in particular 1 to 100 mg, stevioside; in addition to any further components) is present.

For example, the drinks according to the invention can be provided as sodas. In this case, as further preferred additives, e.g. caramel (in the case of caffeine-containing sodas and caffeine-free sodas corresponding to these in flavor note, and also in the case of sodas having apple flavor with or without a fruit juice fraction and clear herbal sodas), caffeine (in the case of caffeine-containing sodas in a fraction of at least 65 milligrams per liter and at most 250 milligrams per liter), whey products, beta-carotene and also riboflavin and coloring foods (except in the case of clear sodas e.g. with citrus aroma), citric acid, extracts of ginger root (e.g. as ginger ale products) or bitter substances (e.g. quinine (from the bark of the cinchona tree; if the soda contains at least 15 mg/l of quinine, it is termed tonic (max. 85 mg/l of quinine))).

A combination with fruit juices, vegetable juices or fruit pulp is also a preferred variant of the drink according to the invention. The finished food can then be provided as fruit juice, fruit juice drink, fruit nectar etc. Further preferred components are the abovementioned; in addition, e.g. lactic acid, citric acid and ascorbic acid can also be added.

Fruit juices or components thereof are generally preserved by heating. A modern process is uperization (made-up word from “ultra” and “pasteurization”). Juice preheated to approximately 80° C. is heated in this process by introducing steam under pressure for some seconds to temperatures between 130 to 150° C. and rapidly cooled again thereafter. Another process is pasteurization at lower temperatures, similar to that with fresh whole milk. In this case the juice is heated to approximately 85° C. for some seconds. A juice preserved in this manner can be kept unrefrigerated for approximately 12-18 months.

According to a further embodiment, the drink according to the invention contains 200 to 1000 mg/l of EGCG and 80 to 350 mg/l of NR or 0.5 to 5 g/l of tryptophan.

As an alternative to the drinks form or to the form as drinks base, such as sachets, effervescent tablets, powder form or concentrates, the composition according to the invention can also be provided as a pharmaceutical formulation in the form of other medicament dosages, e.g. in the form of tablets, capsules, dragees etc. In this case, they can be provided with coatings resistant to gastric juice. Preferably, these medicament dosages are provided in dosage unit form, in particular as daily dose (for example (also in each case independently of one another) 1 mg to 10 g, preferably 1 to 1000 mg, still more preferably 10 to 500 mg, in particular 50 to 300 mg, EGCG, 1 to 500 mg, preferably 10 to 100 mg, in particular 20 to 80 mg, NR and/or 0.05 to 10 g, preferably 0.1 to 5 g, in particular 0.5 to 3 g, tryptophan and also optionally 0.1 mg to 500 mg, preferably 0.5 mg to 200 mg, in particular 1 to 100 mg, stevioside; in addition to other ingredients, such as pharmaceutical carriers or flavor substances). Preferably, the daily doses contain 50 to 200 mg EGCG and 20 to 80 mg NR and/or 0.5 to 3 g tryptophan and also optionally a sweetener, in particular a sweetener comprising stevioside and/or rebaudioside A (preferably in an amount from 1 to 80 mg). The pharmaceutical formulation according to the invention can also be provided, in particular, in the form of a food supplement. Particularly preferred daily doses contain, independently of one another, 80 to 150 mg, in particular 100 to 130 mg, EGCG, 30 to 70 mg, in particular 40 to 60 mg, NR and/or 1 to 3 g tryptophan. These daily doses can of course, as mentioned above, also be provided as a drink, preferably in a volume from 100 to 1000 ml, still more preferably from 200 to 750 ml, in particular 250 to 500 ml.

According to a further aspect, the present invention relates to a combined formulation comprising EGCG and NR (and/or tryptophan; wherein the NR variant is preferred according to the invention) as active ingredients, in particular for use as a composition for concentration enhancement. The combination according to the invention of EGCG and NR has proved particularly useful in the enhancement of concentration. The invention also relates to the use of a combined formulation comprising EGCG and NR as active ingredients, in particular as a composition for concentration enhancement, wherein this combined preparation is present preferably as drink or drink base or as a pharmaceutical formulation as described herein.

Although EGCG can in principle also be taken at a dose of below 1 mg, the activity according to the invention need not then necessarily occur. At a dose of greater than 10 g, although the concentration-enhancing activity is achieved, at such high doses, unwanted side-effects can occur such as, e.g., problems with getting to sleep or other accompanying nervous symptoms. In principle, for regular intake, rather relatively low doses should be chosen; for a single intake to induce a concentration enhancement shortly after, somewhat higher doses not only of ECGC but also of NR (or tryptophan) can be taken. Usually (though this is frequently also dependent on the dose) the concentration-enhancing activity starts about half an hour to one hour after the (oral) intake of the composition according to the invention and is maintained, according to dose, over a relatively long period, e.g. several hours. During this time, the concentration-enhancing activity is expressed not only in the individual performance (i.e. that the individual concentration capacity is enhanced) but also in other concentration parameters, in particular in a delay or prevention of the fall in concentration with time (that is to say in an increase in the mental endurance).

Accordingly, preferred dosages (independently of whether the composition according to the invention is taken as a drink, as a medicament dosage form or in other form) for a single dose (e.g. for rapid induction of an enhanced and longer-lasting concentration ability) are 10 mg to 10 g of EGCG and 1 to 100 mg of NR, preferably 100 mg to 5 g of EGCG and 2 to 50 mg of NR, in particular 500 mg to 4 g of EGCG and 5 to 40 g of NR (or 10 mg to 10 g of EGCG and 0.1 to 10 mg of tryptophan, preferably 100 mg to 5 g of EGCG and 0.5 to 5 g of tryptophan, in particular 500 mg to 4 g of EGCG and 1 to 4 g of tryptophan). Continuous intake of the composition according to the invention can proceed, e.g., once a day, once every two days, once every three days, but individual doses can also be taken several times a day (twice, three times, four times, five times, etc.). In this case it should be ensured that firstly the dose is high enough in order to maintain an elevated level of EGCG and NR (or tryptophan) in the body; and secondly overdoses especially of EGCG should be avoided (see above).

The combined formulation according to the invention can firstly be taken already in combined form (this is in any case also the preferred variant when taken as a drink); on the other hand the combined formulation according to the invention can also be taken in separate form, e.g. the EGCG component can be taken in solid form (e.g. orally as powder, capsules or tablets) and the NR component in the form of an aqueous solution or suspension (e.g. likewise orally) in advance or thereafter. Accordingly, the present invention also relates to a combined formulation comprising EGCG and NR and/or tryptophan as active ingredients, in particular for use as a composition for concentration enhancement, wherein the combined formulation consists of at least two components that can be taken separately from one another and in the one component EGCG is provided and in the other component NR and/or tryptophan is provided.

The invention will be described in more detail on the basis of the examples and figure hereinafter, but without being restricted thereto.

In the drawings:

FIGS. 1 and 2 show the functional relationship between NR, NAD, tryptophan and sirtuins;

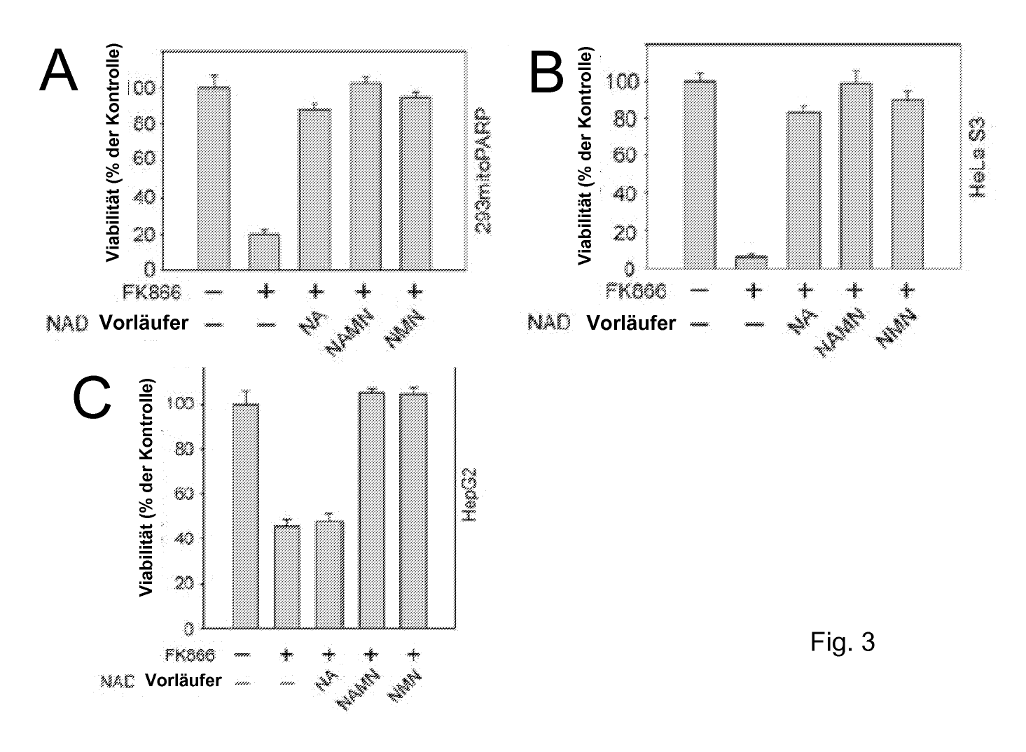

FIG. 3 shows the support of the mitochondrial NAD⁺ generation and survival of the cells by extracellular NAD⁺ derivatives; Nam utilization was inhibited by FK866; both NAMN and NMN support mitochondrial NAD⁺ formation and protect 293mitoPARP (A) and HeLa S3 cells (B) from FK866-induced cell death; mononucleotide precursors, but not NA, support the generation of mitochondrial NAD⁺ and viability of HepG2 cells (C);

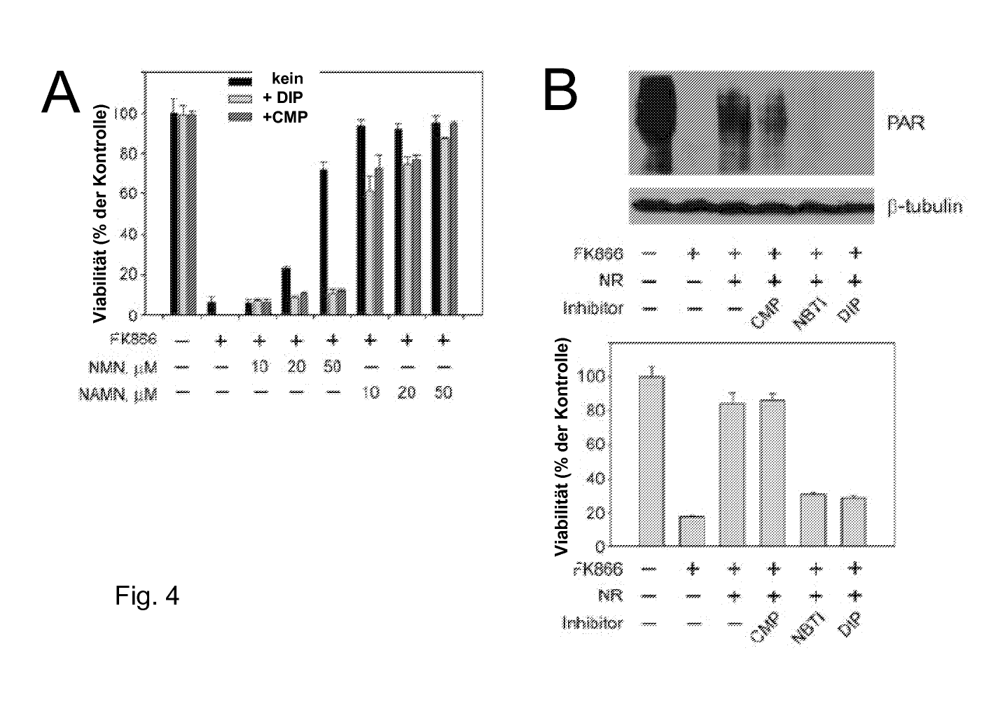

FIG. 4 shows the breakdown of the extracellular nucleotide precursors and the uptake of the resultant ribosides into the cell; when NMN or NANM are used as NAD⁺ precursors, the cell viability depends on the extracellular breakdown of the mononucleotides (A); extracellular NR enters the cell via nucleoside transporters and supports the mitochondrial NAD⁺ generation and cell viability (B);

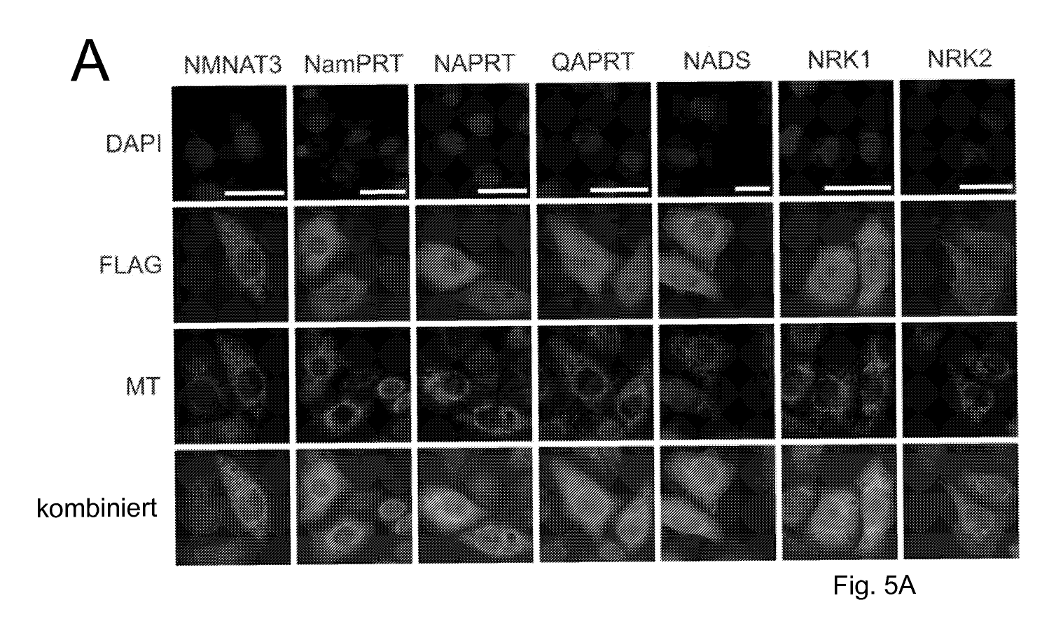

FIG. 5 shows the subcellular localization of the NAD⁺ biosynthesis enzymes; A: all known NAD⁺ biosynthesis enzymes localize in the cytoplasm or nucleus, with the exception of the mitochondrial NMNAT3; C-terminal FLAG-labeled proteins were expressed transiently in HeLa S3 cells; the fluorescence exposures show nuclei (DAPI), expressed recombinant proteins (FLAG) and mitochondria (MT) (bar: 20 μm); B: NMNAT3, but not NamPRT or NAPRT, is a mitochondrial matrix protein; submitochondrial protein localization was carried out after over-expression in HeLa S3 cells using PARAPLAY; PAR accumulation shows matrix localization of the Myc-Tag analyte-PARP1cd fusion protein (bar: 20 μm); and

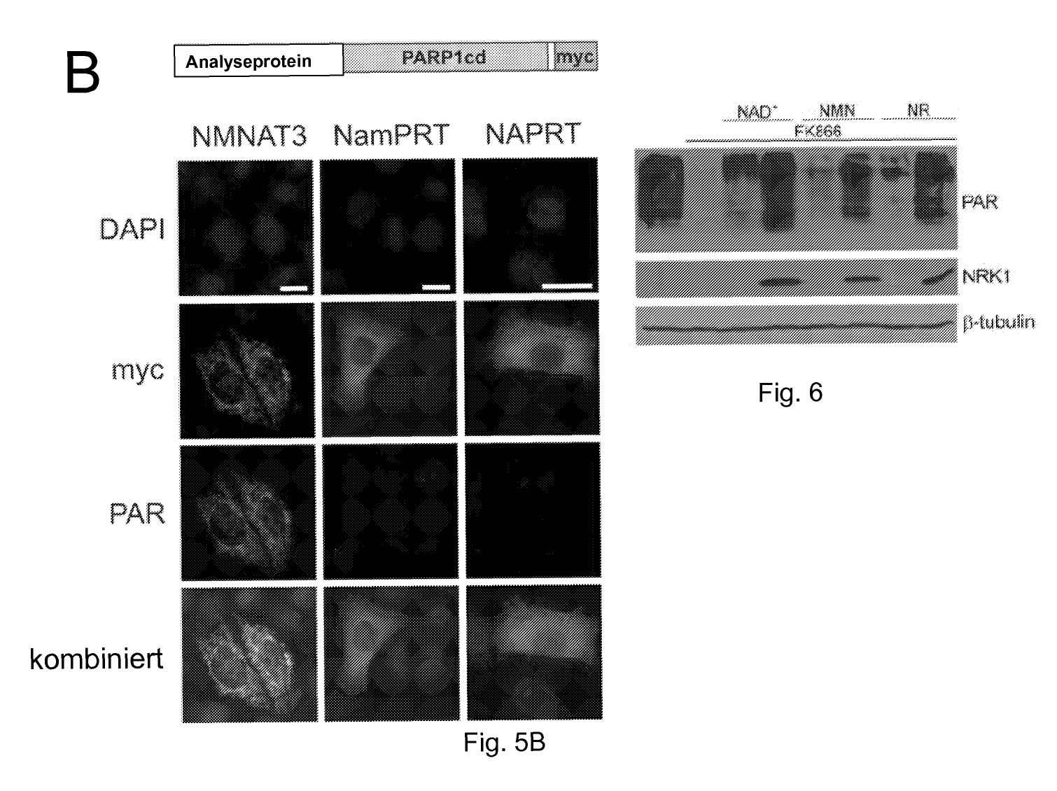

FIG. 6 shows that NMN is the cytosolic precursor of mitochondrial NAD⁺ synthesis; the increase of the mitochondrial NAD⁺ content by overexpression of NRK1 depends on an extracellular NR source (NMN, NAD⁺ or NR, as stated); cytosolic NMN serves as precursor; increased NRK activity increases the mitochondrial NAD⁺ content when NR, NMN or NAD⁺ are provided as extracellular NAD⁺ precursors.

### EXAMPLES

### 1.: Production of the Drink According to the Invention

To one liter of carbonated mountain spring water are added
100 mg of EGCG,
10 mg of NR,
1.2 kg of crystalline citric acid or the amount of liquid citric acid equivalent thereto, and
300 mg of stevia extract
   and are dissolved therein. In this case, about 7 g (approximately 6.8 to approximately 7.2 g) of CO₂ per liter are added to the water.

To one liter of carbonated mountain spring water are added
50 mg of EGCG,
10 mg of NR,
1 g of tryptophan,
1.2 kg of crystalline citric acid or the amount of liquid citric acid equivalent thereto, and
30 mg of stevioside
   and are dissolved therein. In this case, about 7 g (approximately 6.8 to approximately 7.2 g) of CO₂ per liter are added to the water.

To one liter of freshly pressed orange juice are added
500 mg of EGCG,
50 mg of NR,
2 g of tryptophan, and
30 mg of stevioside
   and are dissolved therein.

To one liter of freshly pressed apple juice are added
500 mg of EGCG,
80 mg of NR,
2 g of tryptophan, and
50 mg of stevia extract
   and are dissolved therein. Then, this apple juice mixture is admixed with one liter of carbonated mountain spring water.

To one liter of carbonated mountain spring water are added
200 mg of green tea extract (standardized to 50% EGCG),
10 mg of NR,
1.2 kg of crystalline citric acid or the amount of liquid citric acid equivalent thereto, and
300 mg of stevia extract
   and are dissolved therein.

To one liter of carbonated mountain spring water are added
2 g of EGCG and
20 mg of NR,
   and are dissolved therein.

To one liter of carbonated mountain spring water are added
1 g of EGCG and
50 mg of NR,
   and are dissolved therein.

To one liter of carbonated mountain spring water are added
2 g of EGCG and
80 mg of NR,
   and are dissolved therein.

To one liter of carbonated mountain spring water are added
2 g of EGCG,
50 mg of NR,
1.0 kg of crystalline citric acid or the amount of liquid citric acid equivalent thereto, and
30 mg of stevioside
   and are dissolved therein.

To one liter of carbonated mountain spring water are added
1 g of EGCG,
1 g of green tea extract (standardized to 50% EGCG),
100 mg of NR,
1.0 kg of crystalline citric acid or the amount of liquid citric acid equivalent thereto, and
30 mg of stevioside
   and are dissolved therein.

### 2.: Concentration-Enhancing Activity of the Drink According to the Invention Compared with EGCG Alone

### 2.1.: d2 Test of Attention-Stress and FAIR (Frankfurt Attention Inventory)

The concentration-enhancing activity of the drink according to the invention can be determined especially in comparison with known compositions for concentration enhancement in standard tests for determining concentration. In this case, especially the reinforcing activity of tryptophan and in particular NR is detectable, especially in the particular comparison groups: schoolchildren of the age of from 10 to 18, working women and men from 30 to 40, from 35 to 45, and from 40 to 50, and pensioners from 60 to 70 and from 65 to 75.

The tests used in this case are preferably the d2 test of attention-stress and FAIR (Frankfurt Attention Inventory) (see in addition: e.g. in Westhoff/Hagemeister, “Konzentrationsdiagnostik” [Concentration diagnostics] (2005), pages 41-55).

In this case, for the respective groups of people the subgroups of effective doses of EGCG, NR and/or tryptophan can also be determined for which a statistically significant difference between EGCG alone and the drink according to the invention is just observable in the respective groups.

### 2.2. Triple Test Method for Determining Attention

### 2.2.1. Summary of the Experimental Setup:

Hereinafter the present invention is tested in the context of the Viewpointsystem® vision analyses, using standardized methods of investigation of the Vienna test system and using biomedical studies for stress research. In this method, in the vision laboratory, the triple test method is used with extremely high accuracy for evaluating psychophysical effects up to the degree of attention (cf. EP 1 300 108 A1, WO 2008/151346 A1).

The high significance of the triple test method of Viewpointsystem® is that the combination of the internationally scientifically validated measurement method such as the novel Viewpointsystem® vision research, the psychological Vienna test system and the biomedical test battery guarantees the exact reproducible assessment of the effects of concentration-promoting (performance-boosting) substances.

12 subjects were subdivided into 3 groups each of 4 subjects:

(1) drink according to the invention (150 mg of EGCG/20 mg of NR in 10 ml of H₂O; “energy” or “energy-drink” group
(2) EGCG alone (150 mg of EGCG; “green tea” group)
(3) placebo (H₂O and mannitol; “placebo” group)

### 2.2.2. The Triple Test Study Method:

The subjects were then studied systematically with respect to possible effects using the triple test study method, that is vps vision analyses—Vienna test system—biomedical test battery, in a standardized study, in such a manner that objective, statistically significant statements were made possible on the detection of concentration-enhancing activity of the drink according to the invention.

Using this test system, the changes in attention, vision behavior, reaction, activation and stress were determined in the three groups. In this process the real directions of gaze and gaze motions of subjects were determined in the course of the Vienna test system—in the reaction and determination test, and also in considering defined situation sequences (standardized preset image and film sequences with events) and for selected specific suitability tests—filmed and recorded from the respective field of view (cf. EP 1 300 108 A1, WO 2008/151346 A1).

By documentation in time and space of the gaze movements, the points of view could be clearly documented in motion, wherein, in particular, the onset of gaze and gaze frequencies were able to be detected, standardized and revealed exactly for each situation. By this method, it was therefore possible to work out the differences in the gaze motions, to analyze psychophysical reaction times and gaze fixations and to document optical-physiological gaze latencies precisely. Furthermore, via blinking, the stress parameters could be verified. By this means gaze can be analyzed not only in accordance with place and time (where one is looking for how long), but also the quality of perception can be evaluated. Using the viewpoint high tech programs, the gaze behavior of people can be analyzed with an accuracy of 15 arc minutes or 40 thousandths of a second. This gaze behavior study therefore clarifies exactly all forms of gaze absences, information defects, information deficits, gaze fixations and overinformation (complex situations as the main reasons for making errors, inter alia).

The following are also studied:
accuracy of vision (foveal accuracy)
the differentiation between fixations (points of view) and saccades (jumps of gaze)
the measurement and evaluation of complexity (high requirements) and information density (information in unit time).

In parallel thereto, the relevant biomedical characteristics such as skin conductivity, pulse were also detected and evaluated. In total, therefore, in the course of the triple-test studies, for all action-relevant evaluation situations, gaze studies were carried out in the gaze laboratory, the gaze behavior and reaction times were analyzed. The effects on information uptake, including information stress and fatigue, were documented using the standardized gaze methods by raster-analysis programs.

By documentation of the gaze movements in the sequences performed, physiological performance limits, gaze absences, interfering gaze fixations and other human error frequencies were able to be disclosed and detected exactly. The Viewpointsystem® gaze research combined with detection of the biomedical characteristics allows exact differentiation between changes in behavior and reaction of performance-affected and unaffected subjects and therefore permits an objectivized detection of concentration sequences.

### 2.2.3.: Evaluation Methods (See Also: EP 1 300 108 A1, WO 2008/151346 A1):

As the result, the gaze behavior to detailed depictions with respect to gaze sequence, gaze frequencies according to categories, statements of gaze fixations etc. was recorded, in such a manner that in total an objective evaluation was possible. The determination of complexity, variety and detail recognition was made by FOVEAL PERCEPTION CIRCLES (FPC) gaze films, determination of gaze frequencies and gaze duration (disclosure of the time economy was revealed by dynamic point cloud graphics in the TIME BUBBLES (TB) gaze films. Detailed analyses with following raster analysis programs were provided, wherein the overall results, depending on content, are visualized in a suitable form:

### Use of Raster Analysis Programs:

The viewpointdynamicANALYsis analysis module (according to EP 1 300 108 A1, WO 2008/151346 A1) is currently the most modern analytical method for finding and determining gaze absences and gaze defects (disturbances of ordered information uptake) with simultaneous dynamic depiction of preferably FPC-gaze videos and the associated viewpoint sequences. Using these detailed analyses, the ranges, the high point of interest, high detail recognition values and best time economy have been depicted, compared with those ranges where obviously there is optical-physiologically less interest, low detail recognition and poor time dispositions occurred, or real information errors and information defects were detectable.

The continuous visual depiction of the fixation dominances and saccade dominances was performed using the viewpointcomplexityFINDER on the basis of the priorityzone algorithm in such a manner that the regions of high complexity could be viewed directly in a double-film depiction. By means of this high-tech detection program, the continuous evaluation of the fixation performance and saccade movements (information dropouts, information defects) were able to be made in the film itself. At the same time, the information density (degree of complexity) of the respective section was indicated numerically as a value and visualized particularly clearly via a colored sidebar.

The analyses using the viewpointcomplexityRESEARCH analysis program served for finding and detailed study of positions having information losses, caused by high complexity or frequent foveal central gaze fixations. Using these parameters, therefore, qualitative depth analyses of the real information intake and refined consideration of differing information dropouts and information defects were possible.

Detailed analyses of blinking for determining information stress, the degree of stress and fatigue proceeded using the viewpointeyeblinkRESEARCH program. The blink rate, blink duration, taking particular account of flurries or burst of blinks were able to be shown for determination of the attention rate or the monotony or the fatigue factor.

The continuing comparison and evaluation of the spatial frequency distributions in the visual field were made according to 50%, 85%, 95% distributions with recognition and indication of the priority range. In detailed analyses, the regions, high main interest, high detail recognition values and best time economy were depicted, compared with those ranges where obviously little attention in terms of optical physiology and emotion, low detail recognition and poor time dispositions occur.

### Detailed Evaluation of Physiological Parameters:

Analysis of the stress-causing factors and sequences in real situations is possible using the mobile miniature Physio-Recorder instrument. By this means the activation and stress states of people can be displayed.

The subject carries only a small unit—roughly the size of a wristwatch—and also the electrodes directly on the hand or ear. Here too, a comprehensive display of the bodily reactions is possible with very high accuracy with unrestricted freedom of movement.

After a baseline measurement, the study follows, wherein, inter alia, the following values are recorded:
heart frequency (HF: number of heart beats/minute)
blood volume pulse (BVP: measure of amount of blood which is pumped through the body per heart beat)
skin conductance value (SCL: skin conductance level/conductivity level, tonic measure, SCR: skin conductance response/phasic measure; reaction to individual stimuli)

It is thereby possible to recognize clearly activation processes, adaptation performance and stress situations. By the joint synchronous use of this system and the gaze determination, the connection between the information intake and the bodily reaction is disclosed for the first time, whereby substantially more detailed statements are made on the causes of burdens and stress states or activation states.

### 2.2.4.: Results:

2.2.4.1.: Summary of the Results of the “ENERGY Drink” Study from the Detailed Analyses of the Physiological Parameters

In the detailed analyses, at the same time the gaze and biomedical data of the study sequences [reaction test (singular stimuli), determination test (multiple optical, acoustic stimuli), film viewing (real sequences from driver viewpoint and film trailers) and semiotics (animal pictures and logos)] were evaluated. Particular attention was paid to the skin conductance level (SCL), the heart rate (pulse; HR) and the breathing rate (AF).

These parameters are indications of sympathetic activations of the subjects. The sympathetic nerve system, in addition to the parasympathetic nerve system and the enteric nerve system, is a part of the vegetative nerve system. Most of the organs are controlled by the first two systems, which act as antagonists and thereby permit an extremely fine regulation of the organ activity. The sympathetic nerve system has in this system an ergotropic action, that is to say it increases the outwardly directed readiness to action.

The sympathetic nerve system activates, on the action of stress stimuli, all emergency functions of the body that provide it with readiness to act: pulse and blood pressure increase, the blood glucose level increases, in order to open up a rapidly available energy source, and the attention level is increased. Once the situation has passed, the parasympathetic nervous system gains precedence: pulse and blood pressure slow down, the glucose circulating in the blood falls again. The body is switched over to rest, in order to ensure recuperation for future events.

After the physiological parameters were determined, for each section the statistical parameters mean/standard deviation/min/max/range/85% percentile were determined. In addition, the trend line equations were calculated.

For further statistical detailed evaluation, the 85% percentile values were used and the relative differences were compiled using the before/after relationship. This change was totaled as a dimension number.

After categorization of the subjects into 3 comparison groups, the dimension numbers were summed and represent an index of the physiological/mental stress of the subjects. The lower the number, the “more relaxed” the solving of the tasks by the subjects.

TABLE 1
Classification of the measurements according to subject group

| Energy group [1]: +/+ | | | | Placebo group [2]: −/− | | | | Green tea group [3]: −/+ | | | |
|---|---|---|---|---|---|---|---|---|---|---|---|
| | T1 | T2 | TOTAL | | T1 | T2 | TOTAL | | T1 | T2 | TOTAL |
| PA | 3.12 | 5.03 | 8.2 | PC | 6.08 | 5.52 | 12.45 | PB | 4.81 | 5.45 | 10.25 |
| PE | 4.87 | 5 | 9.87 | PD | 5.35 | 5.44 | 10.79 | PF | 5.51 | 7.02 | 12.53 |
| PG | 6.78 | 6.2 | 12.98 | PK | 5.88 | 5.39 | 11.27 | PH | 6.86 | 5.53 | 12.39 |
| PI | 5.68 | 5.09 | 10.77 | PL | 6.55 | 5.35 | 12.4 | PJ | 5.11 | 5.67 | 10.78 |
| Total | 20.45 | 21.37 | 41.82 | Total | 23.86 | 22.2 | 46.91 | Total | 22.29 | 23.67 | 45.95 |
| Mean | 5.11 | 5.34 | 10.46 | Mean | 5.97 | 5.55 | 11.73 | Mean | 5.57 | 5.92 | 11.49 |

T1: Vienna test system: reaction test/determination test
T2: Viewing film sequences/trailers/logos/animals

TABLE 2
Evaluation units/individual parameters

| | | RT | DT | Film | Animals |
|---|---|---|---|---|---|
| Group 1 | AF | 2.66 | 2.52 | 3.83 | 3.60 |
| | SCL | 4.35 | 4.07 | 3.36 | 3.62 |
| | HR | 3.36 | 3.49 | 3.45 | 3.51 |
| Group 2 | AF | 3.65 | 3.44 | 3.70 | 3.74 |
| | SCL | 5.45 | 5.11 | 3.70 | 3.63 |
| | HR | 3.49 | 3.57 | 3.72 | 3.71 |
| Group 3 | AF | 3.58 | 3.63 | 3.90 | 3.10 |
| | SCL | 4.05 | 4.17 | 4.90 | 4.67 |
| | HR | 3.42 | 3.44 | 3.61 | 3.49 |
| | RT | DT | Film | Animals | Total |
| AF | | | | | |
| Group 1 | 2.66 | 2.52 | 3.83 | 3.60 | 12.61 |
| Group 2 | 3.85 | 3.44 | 3.70 | 3.74 | 14.58 |
| Group 3 | 3.58 | 3.67 | 3.90 | 3.10 | 14.21 |
| SCL | | | | | |
| Group 1 | 4.35 | 4.07 | 3.30 | 3.62 | 15.39 |
| Group 2 | 5.45 | 5.11 | 3.70 | 3.63 | 17.89 |
| Group 3 | 4.05 | 4.17 | 4.90 | 4.67 | 17.79 |
| HR | | | | | |
| Group 1 | 3.36 | 3.49 | 3.45 | 3.51 | 13.82 |
| Group 2 | 3.49 | 3.57 | 3.72 | 3.71 | 14.48 |
| Group 3 | 3.42 | 3.44 | 3.61 | 3.49 | 13.90 |

Evaluation of the sum parameters shows a tendency, especially in the active test sequences (manipulations of the test subjects required), for a higher performance ability for the problem solutions (greater ability to be stressed) of the subjects of the “Energy” and “Green tea” groups. The “Energy” subject group, according to evaluation of the biomedical parameters of breathing frequency, skin conductance level and heart rate, is less stressed.

### 2.2.4.2.: Description of the Detailed Analyses of the Vienna Test System:

### Studies

### Reaction Test (RT)

According to Dorsch (“Psychologisches Worterbuch” [Psychological dictionary], (12th edition) (1994), editors: Häcker et al., Huber Verlag, Bern, Switzerland), “reaction time” is the time which passes between a signal and the start of the mechanical movement response, under the instruction to react as quickly as possible. Since this concerns accuracies in the millisecond range, the test instrument used must be highly reliable and exact. The reaction test, by special test forms, permits accurate measurement of the reaction time and motor time.

### Procedure:

The input medium used is the subject keyboard. An animated instruction phase and an error-sensitive practice phase lead to the formulation of the problem. In the context of the test instruction, color stimuli and acoustic signals are presented to the subjects. The subject receives the instruction to press the reaction key only when relevant stimuli are presented and subsequently to place the finger immediately back on the rest key.

### Determination Test (DT)

The determination test is a particularly accurate measurement method for determining the reactive ability to work under stress and serves for determining the reactive ability to work under stress and also the associated ability to react. The method requires, as cognitive partial performances, the differentiation between various colors and sounds, the conceptual fixing of the relevant features of stimulus configuration and operating elements, and also of the assignment rules, and the choosing of the relevant reaction according to the assignment rules agreed as per instruction and/or learned in the course of the test. The stress factor in the DT is the continuous rapid and variable reaction lasting as long as possible to rapidly changing stimuli.

The application is the measurement of the reactive ability to work under stress, attention and the reaction speed to continuously required rapid and differentiated reactions to rapidly changing optical and acoustic stimuli.

### Procedure

Color stimuli and acoustic signals are presented to the subject. The reaction is performed by actuating the corresponding keys on the subject keyboard. The stimulus presentation proceeds adaptively, the presentation rate is adapted to the performance level of the subject.

On the basis of these results, the gaze studies were then carried out. In addition to the exact measurements of gaze latencies (delayed movement), adaptation parameters such as change speed and duration, the blink rates and blink number were also used for definition of stress parameters.

Results from the Synchronous Gaze Studies

Gaze latencies in real film sequences (delayed gaze movements) are lowest for the Energy subject group for evaluation of real film sequences (real sequence)
best values for this group are shown in the respective after state. Whereas, especially, the Placebo group shows extreme enhancement rates and the green tea group shows low enhancements, the Energy group shows no great enhancements.

The overall evaluation for blink durations and blink number for all test methods (such as reaction test, determination test and real sequences), for the Energy subject group, shows up to 21% shorter blink durations than for the Placebo group and Green tea group. Assuming that the blink duration may be defined as an index of better time-economical action in diverse activities, and at the same time the number of blinks in the Placebo group and the Green tea group is markedly lower, for these two groups a higher stress state can be recognized, and conversely, for the Energy group, an improved management in problem solutions can be interpreted. This result is consistent with the biomedical results from the Vienna test system.

In the evaluation of pupil sizes and change speeds, in detail, the changes during film scenes from dark to light and from light to dark were studied in detail, the pupil sizes compared and the change speeds identified. In this case, the following correlations are found: especially in changes from dark to light, the Energy group has markedly steeper change dimensions, which means that in this group, apparently the eye reacts somewhat more rapidly, in the changes from dark to light, than in the Placebo group. These statements on adaptation tendency also imply an improvement in activation.

In total, these results prove that, using the composition according to the invention, a marked concentration enhancement compared with the comparison groups may be achieved. In particular, this was expressed in an improvement of the mental endurance in the Energy group.

### 3.: Biochemical Characterization

The effectiveness of the composition according to the invention can also be demonstrated biochemically, e.g. by the methods described in Dölle et al. (Anal. Biochem. (385) (2009): 377-379) and Berger et al. (J. Biol. Chem. 43 (280) (2005): 36334-36341).

### 3.1.: Studies on the Subcellular Compartmentation of NAD Biosynthesis in Human Cells:

### 3.1.1.: General:

The concentration of NAD in cells and tissues is of critical importance for maintaining vital functions. Firstly, NAD is a universal energy carrier and is therefore essential for providing ATP, the molecule that supplies the majority of all energy-consuming processes. Secondly, NAD is a cellular signal molecule which participates in the regulation of events important to life by specific conversions. These include, inter alia, regulation of gene expression, cell division, DNA repair and also life span and long-term memory. It is known that increasing the cellular NAD concentration beneficially affects these processes.

In human cells, NAD is synthesized from vitamin B3. Vitamin B3 comprises two very similar substances, nicotinamide and nicotinic acid (nicotinate). Nicotinamide, under usual conditions, is present sufficiently in the diet, and so further increase (e.g. as food supplementation) is virtually without effect. Since nicotinate is converted to NAD via an alternative synthesis pathway, an increased supply would be a promising strategy for increasing NAD. In fact, nicotinate is employed therapeutically as a lipid- (or cholesterol-)lowering agent, where these effects are explained by receptor-mediated mechanisms. These mechanisms also appear to be the causes of considerable side-effects, among which skin irritations, rash and dizziness occur particularly frequently. Therefore, nicotinate is unsuitable in order to be used as a food supplement for increasing NAD.

In the more recent literature, nicotinamide-riboside (NR) has been described as a physiological intermediate of NAD metabolism that is converted to NAD in an alternative pathway.

Hereinafter, experimental findings are presented which make it clear that NR can in fact be taken up by human cells and converted to NAD. Although little is yet known on the content of NR in the diet, there are marked indications that NR is only present there in very small amounts. However, with the experimental findings shown herein, it is demonstrated that the NAD concentration can be increased in tissue using NR, without in this process causing the side-effects of nicotinate. In addition, it has been able to be observed that adding EGCG to human cells can increase by 40% the expression of NMNAT1, an isoform of the enzyme NMNAT (nicotinamide-mononucleotide-adenylyl transferase) which is found in all cells. This also provides a physiological-scientific explanation for the concentration-enhancing action of the drink according to the invention, where apparently the combined administration of NR and EGCG beneficially affects the cellular NAD synthesis by a synergistic effect. Firstly, NR, in addition to the nicotinamide in the diet, is supplied to NAD synthesis as an alternative precursor substance. Secondly, EGCG induces a necessary increase in the capacity of the enzyme which participates in both synthetic pathways.

### 3.1.2.: Experimental Methods:

### Cloning and Generation of Eukaryotic Expression Vectors

For expressing the tagged proteins, the corresponding open reading frames were introduced into PFLAG-CMV-5a (Sigma), pcDNA3.1(+)-PARP1cd (Dölle et al., Cell. Mol. Life Sci. 67 (2010), 433-443) or pCMV/myc/mito (Invitrogen). All cloned DNA sequences were verified by DNA sequence analysis.

### Pharmacological Treatments

Inhibitors (2 μm FK866, 2 mM 3-AB, 10 μm NBTI, 2 μm dipyridamol, 2 mM CMP, 25 μm PPADS, 1 mM Ap4A) or metabolites (100 μM NAD⁺, 100 μM NAAD, 100 μM NMN, 100 μM NAMN, 100 μM NA, 100 μM NR) were added to the cell culture medium as stated. NR was prepared as described (Dölle et al., Anal, Biochem. 385 (2009), 377-379). The treatment with inhibitors and metabolites was carried out for 24-48 h before analysis of PAR formation and the viability of the cells (72 h for 293mitoPARP cells, 96 h for HeLa S3 cells and 120 h for HepG2 cells). The viability of the cells was determined by MTT assay.

### Immunocytochemistry

The cells were fixed with 4% (v/v) formaldehyde in PBS and permeabilized using 0.5% (v/v) Triton X-100 in PBS. Cell nuclei were stained with DAPI and mitochondria with MitoTracker Red CMXRos (Invitrogen). Images were taken with a Leica DMI6000B epifluorescence microscope (Leica Microsystems) equipped with ×10, ×40 and ×100 lenses.

### Detection of Changes in the NAD⁺ Content by PARP Activity in Mitochondria

293mitoPARP cells were prepared that express a fusion protein (“mitoPARP”), consisting of EGFP and PARP1cd (poly-ADP-ribose polymerase-1) which was targeted to the mitochondrial matrix. With mitochondrial NAD⁺ as substrate, these cells generate protein-bound PAR (protein-bound poly-ADP ribose) constitutively, which is visualized immunochemically. Variations in the extent of the PAR detected thereby reflect changes in the mitochondrial NAD⁺ content. Likewise, the transient expression of mitoPARP in HepG2 and HeLa S3 cells was used.

### Identification of Mitochondrial Matrix Proteins by PARAPLAY

The Poly-ADP Ribose Assisted Protein Localization Assay (PARAPLAY) delivers a luminal protein localization (Dölle et al., 2010). Here, PARAPLAY was used for identification of mitochondrial matrix proteins. The proteins that were to be analyzed (NMNAT3, NamPRT and NAPRT) were expressed in HeLa S3 cells as N-terminal fusion proteins with PARP1cd. On account of the low [NAD⁺] and/or the high PAR-degrading activity in the cytosol and in the mitochondrial intermembrane space, no PAR was able to be detected when the fusion protein is present in these regions. In contrast thereto, matrix localization is readily detectable by PAR accumulation. Even if the majority of the fusion protein is present in the cytosol, a luminal fraction of the protein is sufficient to generate sufficiently immunologically detectable PAR. If no PAR can be detected, the localization of the protein outside the matrix is verified by testing the functionality of the PARP1cd part of the fusion protein. This is achieved by adding an N-terminal mitochondrial targeting sequence to the fusion protein, whereby it is passed into the matrix and leads to PAR accumulation.

### 3.1.3.: Results:

### In Situ Detection of Relative Mitochondrial NAD⁺ Levels by Poly-ADP Ribose Generation in the Matrix

A targeted expression of PARP1cd in the mitochondrial matrix, here fused to EGFP and termed “mitoPARP”, leads to a constitutive presence of PAR within these organelles. A PAR signal depends not only on the catalytic activity of mitoPARP but also on the presence of NAD⁺. Consequently, the mitoPARP expression permits the recognition of changes in mitochondrial NAD⁺ content. PAR was not detectable in any of the experiments in the cell nucleus, the site at which endogenous PARP-1 is localized.

Identification of Extracellular NAD⁺ Metabolites which Support Mitochondrial NAD⁺ Generation

A study was then made of what extracellular NAD⁺ precursors support mitochondrial NAD⁺ generation. In each case Nam fulfills this function because it is generally the only NAD⁺ precursor in cell culture media. If, therefore, NamPRT is inhibited, an alternative precursor must be present for the NAD⁺ generation. Addition of Na, NAMN or NMN to the medium was able to restore the mitochondrial NAD⁺ content and the cell survival (FIG. 3A). The viability of the cells correlates with the mitochondrial NAD⁺ level, in accordance with PAR measurement. Transfected HeLa S3 cells showed a similar sensitivity to FK866 and restoration of survival by NA, NAMN and NMN (FIG. 3B). In contrast thereto, NA in HepG2 cells was not able to provide support for the generation of NAD⁺ and the survival of the cell (FIG. 3C), which confirmed the lack of NA-phosphoribosyl transferase (NAPRT) activity in this cell line.

Extracellular nucleotides are broken down to the corresponding nucleosides which then enter into the cells as NAD⁺ precursors

The NPP inhibitor PPADS prevents the restoration of the mitochondrial NAD⁺ pool and the survival of the cell when NAD⁺ was used as extracellular precursor. Likewise, the addition of diadenosine tetraphosphate (Ap4A); as a competitive NPP substrate, led to a similar effect on the viability of the cells not only in 293mitoPARP but also in HeLa S3 cells. In order therefore to serve as a precursor, extracellular NAD⁺ must be broken down to NMN. The addition of CMP (competes with NMN as substrate for the dephosphorylation by external 5′-nucleotidase) decreases the cell viability of 293mitoPARP and HeLa S3 cells markedly. Further, NMN lost its function as an extracellular NAD⁺ precursor also in the presence of dipyridamol and NBTI, both inhibitors of the plasma membrane nucleoside transporter. The mitochondrial NAD⁺ generation of NAMN is also sensitive to these inhibitors, although less strongly (FIG. 4A). This inhibition could be overcome by increasing the concentration of NAMN.

NR supports the mitochondrial PAR formation and cell viability in the presence of CMP, while dipyridamol and NBTI inhibit the NR usage (FIG. 4B). From these results, it can therefore be concluded that, in addition to NA and Nam, only the riboside precursors NR and NAR act as extracellular precursors of intracellular NAD⁺, while mono- (NMN and NAMN) and dinucleotides (NAD⁺ and NAAD) must first be processed to the corresponding nucleosides.

All NAD⁺ biosynthesis enzymes localize in the cytoplasm or nucleus, with the exception of a mitochondrial NMNAT isoform

For understanding of the intracellular signal paths of NAD⁺ biosynthesis, comprehensive analyses of the subcellular distribution of the human enzymes were carried out. When these enzymes are expressed in HeLa S3 cells with a C-terminal FLAG epitope, all enzymes, except for NMNAT3, localize in the cytoplasm or in the cell nucleus (FIG. 5A). NMNAT3 co-localizes with Mitotracker; for the two other NMNAT isoforms, it is known that they localize at the nuclear or cytosolic side of the Golgi apparatus.

Both NamPRT and NAPRT generate substrates for NMNATs. If these were contained in the present matrix, they could contribute to the mitochondrial NAD⁺ synthesis, assuming NMNAT3 is a matrix protein. In order to clarify this question, the PARAPLAY assay was employed, which is especially suitable for resolving suborganelle protein localization (Dölle et al., 2010). This method comprises the overexpression of an analyte protein which is fused to PARP1cd. For mitochondrial proteins, PAR accumulation is only observed when the fusion protein is situated within the matrix. Therefore, NMNAT3, NamPRT and NAPRT were expressed as fusion proteins with PARP1cd. The localization of these constructs (FIG. 5B) was similar to the corresponding FLAG proteins (FIG. 5A): the NMNAT3-PARP1cd protein supports PAR accumulation in mitochondria, which likewise verifies that NMNAT3 is in fact a matrix protein. Neither the NamPRT- nor the NAPRT-PARP1cd fusion protein gave a detectable polymer formation, which verifies that they are not proteins of the mitochondrial matrix in human cells.

It follows therefrom that NMNAT3 is the sole enzyme of NAD⁺ synthesis that is present in mitochondria of human cells. All other enzymatic activities of human NAD⁺ biosynthesis are present in the nucleus and/or the cytoplasm. Therefore, it is clear that nuclear/cytosolic NAD⁺ can be synthesized from NR as an extracellular precursor.

### NMN is the Cytosolic Precursor of Mitochondrial NAD⁺ Synthesis

The presence of only NMNAT3 in the matrix points strongly to NMN as cytosolic precursor of mitochondrial NAD⁺. The absence of NAD synthetase (NADS) in mitochondria excludes the possibility of amidating NA precursors within the organelles. Furthermore, the localization of both NRK isoforms outside mitochondria indicates that the phosphorylation of NR does not occur within the organelles.

After entry into the cell, NR must be into NMN in the cytosol. In fact, overexpression of NRK1 (which is cytoplasmic) leads to a dramatic increase of the amount of mitochondrial PAR, but only when an extracellular precursor (such as NMN) was available. Therefore, NR is a potent precursor of mitochondrial NAD⁺ when it is phosphorylated in the cytosol to NMN. Accordingly, NMN was thereby functionally confirmed as the cytosolic precursor of mitochondrial NAD⁺. The overexpression of NRK1 considerably improved the use of extracellular NAD⁺ or NR itself for mitochondrial PAR formation (FIG. 6). These observations confirm that extracellular NMN and NAD⁺ are not taken up into the cells, but rather must first be broken down to NR.

This also explains the particularly rapid action of NR in the context of the present invention in the synergistic activity with EGCG in the activation of these cellular processes.

1-12. (canceled)

13. A drink, comprising, per liter:
1 mg to 10 g epigallocatechin gallate; and
1 to 500 mg nicotinamide-ribose, and/or 0.05 to 10 g tryptophan.

14. The drink according to claim 13, wherein the epigallocatechin gallate is present in an amount from 1 to 1000 mg, and/or the nicotinamide-ribose is present in an amount from 10 to 100 mg, and/or the tryptophan is present in an amount from 0.1 to 5 g.

15. The drink according to claim 13, wherein the epigallocatechin gallate is present in an amount from 10 to 500 mg, and/or the nicotinamide-ribose is present in an amount from 20 to 80 mg, and/or the tryptophan is present in an amount from 0.5 to 3 g.

16. The drink according to claim 13, wherein the epigallocatechin gallate is present in an amount from 50 to 300 mg.

17. The drink according to claim 13, further comprising an amount of stevioside.

18. The drink according to claim 17, wherein the stevioside is present in an amount from 0.1 to 500 mg.

19. The drink according to claim 17, wherein the stevioside is present in an amount from 1 to 100 mg.

20. The drink according to claim 13, present in volume from 100 ml to 2 liters.

21. A drink base, comprising a mixture of components forming a base which, upon the addition of a liquid selected from the group consisting of water, mineral water, and fruit juice forms the drink according to claim 13.

22. The drink base according to claim 21, where said mixture is present in solid form.

23. The drink base according to claim 22, where said mixture is in powder form.

24. A pharmaceutical formulation in dosage unit form, comprising:
1 mg to 10 g epigallocatechin gallate;
1 to 500 mg nicotinamide-ribose; and/or 0.05 to 10 g tryptophan.

25. The pharmaceutical formulation according to claim 24, wherein the epigallocatechin gallate is present in an amount from 1 to 1000 mg, and/or the nicotinamide-ribose is present in an amount from 10 to 100 mg, and/or the tryptophan is present in an amount from 0.1 to 5 g.

26. The pharmaceutical formulation according to claim 24, wherein the epigallocatechin gallate is present in an amount from 10 to 500 mg, and/or the nicotinamide-ribose is present in an amount from 20 to 80 mg, and/or the tryptophan is present in an amount from 0.5 to 3 g.

27. The pharmaceutical formulation according to claim 24, wherein the epigallocatechin gallate is present in an amount from 50 to 300 mg.

28. A combined formulation, comprising epigallocatechin gallate and nicotinamide-ribose as active ingredients.

29. The combined formulation according to claim 28, wherein the EGCG is present in an amount from 10 mg to 10 g and the NR is present in an amount from 1 to 100 mg.

30. The combined formulation according to claim 28, wherein the EGCG is present in an amount from 100 mg to 5 g and the NR is present in an amount from 2 to 50 mg.

31. The combined formulation according to claim 28, wherein the EGCG is present in an amount from 500 mg to 4 g and the NR is present in an amount from 5 to 40 mg.

32. A combined preparation, comprising epigallocatechin gallate and nicotinamide-ribose and/or tryptophan as active ingredients, suitable in a composition for concentration enhancement, wherein the combined preparation consists of at least two components that can be taken separately from one another, with a first component being EGCG and a second component being NR and/or tryptophan.
